(19) United States
(12) Patent Application Publication
(10) Pub. No.: US 2016/0222305 A1
(43) Pub. Date: Aug. 4, 2016

(54) METHODS AND COMPOSITIONS FOR DECREASING FOULING WITHIN AN ETHYLENE PLANT

(71) Applicant: BAKER HUGHES INCORPORATED, Houston, TX (US)

(72) Inventors: Hua Mo, Friendswood, TX (US); Roger D. Metzler, Sugar Land, TX (US); Jonathan R. Bui, The Woodlands, TX (US)

(73) Assignee: BAKER HUGHES INCORPORATED, Houston, TX (US)

(21) Appl. No.: 14/610,688

(22) Filed: Jan. 30, 2015

(51) Int. Cl. C10G 75/04; C07C 11/04

(52) U.S. Cl. CPC C10G 75/04; C07C 11/04

(57) ABSTRACT

Methods and fluid compositions are described for decreasing or inhibiting foulants within an ethylene plant at a particular location, such as a caustic tower in a non-limiting embodiment. An effective amount of at least one substituted hydroxylamine and an effective amount of at least one aldol inhibitor may be introduced into a fluid feed of the ethylene plant. The aldol inhibitor(s) may be or include a substituted hydroxylimine, a borohydride, and combinations thereof.

### TECHNICAL FIELD

The present invention relates to decreasing or inhibiting foulants within a fluid feed of an ethylene plant, and more specifically relates to introducing at least one substituted hydroxylamine and at least one aldol inhibitor into the fluid feed at the same time or different times.

### BACKGROUND

A cracking furnace of an ethylene plant may produce many foulants as the ethylene and other useful olefins are produced. Such foulants may be acid gases, carbon dioxide (CO₂), hydrogen sulfide (H₂S), and to a lesser extent some of the weaker acid gases such as mercaptans (R—SH), which must be removed efficiently to prevent negative consequences of the foulants flowing into downstream catalyst beds.

The hydrogen sulfide content may be the result of two sulfur sources associated with the pyrolysis step in an ethylene production process. First, sulfur may be naturally occurring in an ethylene plant's fresh hydrocarbon cracking feed. Second, sulfur may be deliberately flowed into furnace radiant coils, which is also associated with the carbon dioxide production in the cracking furnaces.

Various types of feedstocks may be part of the fluid stream within an ethylene plant. Each feedstock may have various types of foulants therein. The feedstocks may be or include, but are not limited to, liquefied petroleum gas (LPG) feedstocks used for gas cracking, caustic treated C₃ LPG feedstocks may have carbonyl sulfide (COS) and mercaptans, caustic treated C₄ feedstocks may have light mercaptans (e.g. 10 to 50 ppm by weight), light naphthas or natural gas liquids may have heavy mercaptans (e.g. 100 to 150 ppm by weight), low sulfur full range napthas with straight chain organic compounds, atmospheric gas oil feedstocks, heavy atmospheric gas oil feedstocks, vacuum gas oil feedstocks, field condensates (a prime cracking feedstock for many feed flexible liquid crackers), and combinations thereof.

A caustic tower within the ethylene plant removes the foulants present in the fluid feed by using a dilute caustic soda to absorb the foulants. Decreasing the amount of foulants present in the fluid feed is a key factor in obtaining ethylene and propylene production. The tight limit on the allowable contaminant content levels driven by polymer producers necessitates close scrutiny and evaluation of the operation and performance of the caustic tower system.

Thus, it would be desirable if new additives could be developed to decrease fouling within an ethylene plant, and/or a fluid feed circulating therethrough.

### SUMMARY

There is provided, in one form, a method for decreasing foulants in a fluid feed within an ethylene plant by introducing an effective amount of at least one substituted hydroxylamine and an effective amount of at least one aldol inhibitor into the fluid feed to decrease an amount of foulants present within the fluid feed. The aldol inhibitor(s) may be or include, but are not limited to, a substituted hydroxylimine, a borohydride, and combinations thereof.

There is further provided in another non-limiting embodiment, a fluid composition within an ethylene plant that may include a fluid feed, an effective amount of at least one substituted hydroxylamine, and an effective amount of at least one aldol inhibitor. The fluid composition may have a decreased amount of foulants present within the fluid feed as compared to an otherwise identical fluid feed absent the effective amount of the substituted hydroxylamine(s) and/or the effective amount of the aldol inhibitor(s). The aldol inhibitor(s) may be or include, but are not limited to, a substituted hydroxylimine, a borohydride,and combinations thereof.

The additive appears to at least partially inhibit the polymerization of the polymerizable monomers over a period of time.

### DETAILED DESCRIPTION

It has been discovered that foulants within an ethylene plant may be decreased and/or inhibited by introducing an effective amount of at least one substituted hydroxylamine and an effective amount of at least one aldol inhibitor into a fluid feed of the ethylene plant. In addition or in the alternative, the substituted hydroxylamine(s) and aldol inhibitor(s) may inhibit free radical polymerization, aldol condensation reactions, and combinations thereof. The foulants may be or include, but are not limited to carbon dioxide, hydrogen sulfide, mercaptans, and combinations thereof.

The effective amount of the substituted hydroxylamine(s) may range from about 0.0001 ppm independently to about 10,000 ppm, alternatively from about 0.01 ppm independently to about 5,000 ppm. As used herein with respect to a range, “independently” means that any threshold may be used together with another threshold to give a suitable alternative range, e.g. about 0.0001 ppm independently to about 0.01 ppm is also considered a suitable alternative range.

The substituted hydroxylamine(s) may be represented by Formula (I):

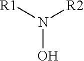

where R₁ and R₂ may have or include an alkyl group, an aromatic group, a hydrogen, a repeatable unit, and combinations thereof. R₁ and R₂ may be the same or different.

Non-limiting examples of the substituted hydroxylamine may be or include, but are not limited to, hydroxylamines substituted with at least one alkyl group, an aryl group, or an alkylaryl group; diethylhydroxylamine; hexapentahydroxylamine; N-ethylhydroxylamine (EHA); N,N′-diethylhydroxylamine (DEHA); N-ethyl-N-methylhydroxylamine (EMHA); N-isopropylhydroxylamine (IPHA); N,N′-dibutylhydroxylamine (DBHA); N-amylhydroxylamine (AHA); N-phenylhydroxylamine (PHA); and combinations thereof.

The effective amount of the aldol inhibitor(s) may range from about 0.0001 ppm independently to about 10,000 ppm, alternatively from about 0.01 ppm independently to about 5,000 ppm. The aldol inhibitor(s) may be or include, but are not limited to, a substituted hydroxylimine, a borohydride, and combinations thereof. The substituted hydroxylimine may be represented by Formula (II):

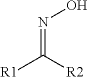

where R₁ and R₂ may be or include an alkyl group, an aromatic group, a hydrogen, and combinations thereof. R₁ and R₂ may be the same or different.

The borohydride may be or include, but is not limited to, an alkali earth metal borohydride, such as a sodium borohydride in a non-limiting embodiment. A non-limiting example of a substituted hydroxylimine is oxime.

The molar ratio of the substituted hydroxylimine(s) to the aldol inhibitor(s) may range from about 1:1,000 independently to about to about 1,000:1, alternatively from about 1:500 independently to about to about 500:1. The substituted hydroxylamine(s) may be introduced at the same time or different time from the aldol inhibitor(s). The substituted hydroxylamine(s) and/or the aldol inhibitor(s) may be introduced into the fluid stream at a location, such as but not limited to, in a caustic tower, prior to the caustic tower, after the caustic tower, and combinations thereof. In a non-limiting instance, the substituted hydroxylamines and/or aldol inhibitors may be added to the fluid stream at a location that is directly prior to the caustic tower, into the caustic tower, directly after the caustic tower, and combinations thereof.

‘Directly prior’ is defined herein to mean the additive or its individual components may be added to the fluid stream prior to the caustic tower, but the caustic tower is the next piece of equipment into which the fluid stream may flow. Similarly, ‘directly after’ is defined herein to mean that the additives or its individual components may be added to the fluid stream as the fluid stream exits the caustic tower.

In a non-limiting embodiment, an effective amount of at least one water-based dispersant may be introduced into the fluid feed of an ethylene plant in an amount ranging from about 0.0001 ppm independently to about 10,000 ppm, alternatively from about 0.01 ppm independently to about 5,000 ppm. The water-based dispersant(s) may be or include, but are not limited to, a polyether dispersant, a polyethylene glycol dispersant, a long chain amide surfactant, a fatty acid surfactant, and combinations thereof.

The water-based dispersant(s) may be introduced at the same time or different time from the substituted hydroxylamine(s) and/or the aldol inhibitor(s). The water-based dispersants may be introduced into the fluid stream at a location of the ethylene plant, such as but not limited to, a caustic tower, prior to the caustic tower, after the caustic tower, and combinations thereof. In a non-limiting instance, the water-based dispersant may be added to the fluid stream at a location that is directly prior to the caustic tower, into the caustic tower, directly after the caustic tower, and combinations thereof.

Introducing the substituted hydroxylamine(s) and the aldol inhibitor(s) into a fluid feed may form a treated fluid composition. The treated composition may have a decreased amount of foulants present within the fluid feed as compared to an otherwise identical fluid feed absent the effective amount of the substituted hydroxylamine(s) and/or effective amount of aldol inhibitor(s). ‘Fluid feed’ as defined herein refers to a feed having a liquid-based stream, a gas-based stream, or a combination thereof.

The methods described are considered successful if the substituted hydroxylamine(s), aldol inhibitor(s), and optional water-based dispersant(s) decrease an amount of fouling than would occur in the absence of these components. Alternatively, success is obtained if a majority of the fouling is decreased or inhibited or inactivated, i.e. at least 51%, or from about 70% independently to about 99.9%, or from about 90% independently to about 96% in another non-limiting embodiment.

The substituted hydroxylamine(s), the aldol inhibitor(s), and the optional water-based dispersant(s) may be dispersed in a suitable liquid carrier dispersing medium, such as but not limited to, water, dimethylformaldehyde (DMF), dimethyl sulfoxide (DMSO), and combinations thereof. The amount of the solvent used with the additive may have a ratio based on weight ranging from about a 100:1 ratio independently to about a 2:1 ratio, alternatively from about a 20:1 ratio independently to about a 2:1 ratio.

The additive, or the individual components (i.e. the substituted hydroxylamine(s), the aldol inhibitor(s), and the optional water-based dispersant(s)) may be directly introduced or injected into the fluid feed. In one non-limiting embodiment, the additive or individual components may be injected into the fluid feed on a continuous basis. Alternatively, the additive or individual components may be injected about every 0.5 hour to about 1 hour in a non-limiting embodiment embodiment.

In the foregoing specification, the invention has been described with reference to specific embodiments thereof, and has been described as effective in providing methods and fluid compositions for decreasing foulants in a fluid stream of an ethylene plant. However, it will be evident that various modifications and changes can be made thereto without departing from the broader spirit or scope of the invention as set forth in the appended claims. Accordingly, the specification is to be regarded in an illustrative rather than a restrictive sense. For example, substituted hydroxylamines, substituted hydroxylimines, foulants, water-based dispersants, and solvents falling within the claimed parameters, and specific proportions or dosages, but not specifically identified or tried in a particular composition or method, are expected to be within the scope of this invention.

The present invention may suitably comprise, consist or consist essentially of the elements disclosed and may be practiced in the absence of an element not disclosed. For instance, the method for decreasing foulants in a fluid feed within an ethylene plant may consist of or consist essentially of introducing an effective amount of at least one substituted hydroxylamine and an effective amount of at least one aldol inhibitor into the fluid feed to decrease an amount of foulants present within the fluid feed; the aldol inhibitor(s) may be or include, but are not limited to, a substituted hydroxylimine, a borohydride, and combinations thereof.

The composition may include a fluid feed, an effective amount of at least one substituted hydroxylamine, and an effective amount of at least one aldol inhibitor; the fluid composition may have a decreased amount of foulants present within the fluid feed as compared to an otherwise identical fluid feed absent the effective amount of the substituted hydroxylamine(s) and/or the effective amount of the aldol inhibitor(s); the aldol inhibitor(s) may be or include, but are not limited to, a substituted hydroxylimine, a borohydride, and combinations thereof.

The words “comprising” and “comprises” as used throughout the claims, are to be interpreted to mean “including but not limited to” and “includes but not limited to”, respectively.

What is claimed is:

1. A method for decreasing foulants in an ethylene plant; wherein the method comprises:
introducing an effective amount of at least one substituted hydroxylamine into a fluid feed of the ethylene plant;
introducing an effective amount of at least one aldol inhibitor into the fluid feed of the ethylene plant; wherein the at least one aldol inhibitor is selected from the group consisting of a substituted hydroxylimine, a borohydride, and combinations thereof, and
decreasing an amount of foulants present in the fluid feed.

2. The method of claim 1, wherein the foulants are selected from the group consisting of carbon dioxide, hydrogen sulfide, mercaptans, polyolefins, polyaromatics, carbonyl derivatives, and combinations thereof.

3. The method of claim 1, wherein the substituted hydroxylamine is represented by the formula: 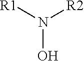
wherein R₁ and R₂ are selected from the group consisting of an alkyl group, an aromatic group, a hydrogen, a repeatable unit, and combinations thereof; and wherein R₁ and R₂ are the same or different.

4. The method of claim 1, wherein the substituted hydroxylimine is represented by the formula: 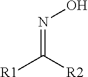
wherein R₁ and R₂ are selected from the group consisting of an alkyl group, an aromatic group, a hydrogen, and combinations thereof; and wherein R₁ and R₂ are the same or different.

5. The method of claim 1, wherein the effective amount of the at least one substituted hydroxylamine ranges from about 0.0001 ppm to about 10,000 ppm.

6. The method of claim 1, wherein the effective amount of the at least one aldol inhibitor ranges from about 0.0001 ppm to about 10,000 ppm.

7. The method of claim 1, wherein a molar ratio of the at least one substituted hydroxylamine to the at least one aldol inhibitor ranges from about 1:1000 to about 1000:1.

8. The method of claim 1, further comprising introducing at least one water-based dispersant into the fluid feed of the ethylene plant; wherein the at least one water-based dispersant is selected from the group consisting of a polyether dispersant, a polyethylene glycol dispersant, a long chain amide surfactant, a fatty acid surfactant, and combinations thereof.

9. The method of claim 8, wherein the amount of the at least one water-based dispersant ranges from about 0.0001 ppm to about 10,000 ppm.

10. The method of claim 1, wherein the introducing the at least one substituted hydroxylamine and/or the at least one aldol inhibitor occurs in a caustic tower of the ethylene plant.

11. A method for decreasing foulants in an ethylene plant; wherein the method comprises:
introducing an effective amount of at least one substituted hydroxylamine into a fluid feed of the ethylene plant; wherein the substituted hydroxylamine is represented by the formula: 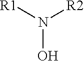
wherein R₁ and R₂ are selected from the group consisting of an alkyl group, an aromatic group, a hydrogen, a repeatable unit, and combinations thereof; and wherein R₁ and R₂ are the same or different
introducing an effective amount of at least one aldol inhibitor into the fluid feed of the ethylene plant; wherein the at least one aldol inhibitor is selected from the group consisting of a substituted hydroxylimine, an alkali earth metal borohydride, and combinations thereof; wherein the substituted hydroxylimine is represented by the formula: 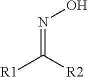
wherein R₁ and R₂ are selected from the group consisting of an alkyl group, an aromatic group, a hydrogen, and combinations thereof; and wherein R₁ and R₂ are the same or different; and
decreasing an amount of foulants present in the fluid feed.

12. A fluid composition within an ethylene plant comprising:
a fluid feed;
an effective amount to decrease foulants of at least one substituted hydroxylamine;
an effective amount to decrease foulants of at least one aldol inhibitor; wherein the at least one aldol inhibitor is selected from the group consisting of a substituted hydroxylimine; a borohydride, and combinations thereof; and
wherein a decreased amount of foulants are present within the fluid feed as compared to an otherwise identical fluid feed absent the effective amount of the at least one substituted hydroxylamine and/or the effective amount of the at least one aldol inhibitor.

13. The fluid composition of claim 12, wherein the foulants are selected from the group consisting of carbon dioxide, hydrogen sulfide, mercaptans, polyolefins, polyaromatics, carbonyl derivatives, and combinations thereof.

14. The fluid composition of claim 12, wherein the substituted hydroxylamine is represented by the formula: 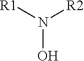
wherein R₁ and R₂ are selected from the group consisting of an alkyl group, an aromatic group, a hydrogen, and combinations thereof; and wherein R₁ and R₂ are the same or different.

15. The fluid composition of claim 12, wherein the substituted hydroxylimine is represented by the formula: 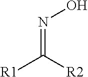
wherein R₁ and R₂ are selected from the group consisting of an alkyl group, an aromatic group, a hydrogen, a repeatable unit, and combinations thereof; and wherein R₁ and R₂ are the same or different.

16. The fluid composition of claim 12, wherein the effective amount of the at least one substituted hydroxylamine ranges from about 0.0001 ppm to about 10,000 ppm.

17. The fluid composition of claim 12, wherein the effective amount of the at least one aldol inhibitor ranges from about 0.0001 ppm to about 10,000 ppm.

18. The fluid composition of claim 12, wherein a molar ratio of the at least one substituted hydroxylamine to the aldol inhibitor ranges from about 1:1000 to about 1000:1.

19. The fluid composition of claim 12 further comprising at least one water-based dispersant; wherein the at least one water-based dispersant is selected from the group consisting of a polyether dispersant, a polyethylene glycol dispersant, a long chain amide surfactant, a fatty acid surfactant, and combinations thereof.

20. The fluid composition of claim 19, wherein the amount of the at least one water-based dispersant ranges from about 0.0001 ppm to about 10,000 ppm.
